(19) United States
(12) Patent Application Publication
(10) Pub. No.: US 2015/0219737 A1
(43) Pub. Date: Aug. 6, 2015

(54) MAGNETIC RESONANCE RADIOFREQUENCY ANTENNA UNIT AND A MAGNETIC RESONANCE DEVICE HAVING THE LOCAL MAGNETIC RESONANCE RADIOFREQUENCY ANTENNA UNIT, AS WELL AS A METHOD FOR CALCULATING ATTENUATION VALUES OF A LOCAL MAGNETIC RESONANCE RADIOFREQUENCY ANTENNA UNIT FOR A MAGNETIC RESONANCE EXAMINATION COMBINED WITH A PET EXAMINATION

(71) Applicant: SIEMENS AKTIENGESELLSCHAFT, Munich (DE)

(72) Inventors: Matthias FENCHEL, Erlangen (DE); Ralf LADEBECK, Erlangen (DE)

(21) Appl. No.: 14/327,625

(22) Filed: Jul. 10, 2014

(30) Foreign Application Priority Data
Jul. 23, 2013 (DE) 102013214381.8

(51) Int. Cl. G01R 33/56; G01R 33/34; A61B 6/03; A61B 6/00; A61B 5/055; G01R 33/48; G01R 33/385

(52) U.S. Cl. CPC G01R 33/56; G01R 33/481; G01R 33/34; G01R 33/385; A61B 6/4417; A61B 5/055; A61B 6/037; A61B 2576/00

(57) ABSTRACT

A magnetic resonance radiofrequency antenna unit is disclosed, including at least one antenna element and an antenna housing enclosing the antenna element. In at least one embodiment, the magnetic resonance radiofrequency antenna unit includes at least one marking element, wherein the at least one marking element is embodied to transmit magnetic resonance signals during a magnetic resonance measurement.

### PRIORITY STATEMENT

The present application hereby claims priority under 35 U.S.C. §119 to German patent application number DE 102013214381.8 filed Jul. 23, 2013, the entire contents of which are hereby incorporated herein by reference.

### FIELD

At least one embodiment of the invention generally relates to a magnetic resonance radiofrequency antenna unit and/or a magnetic resonance device having the local magnetic resonance radiofrequency antenna unit, and/or a method for calculating attenuation values of a local magnetic resonance radiofrequency antenna unit for a magnetic resonance examination combined with a pet examination.

### BACKGROUND

Local magnetic resonance radiofrequency antenna units are frequently employed for acquiring radiofrequency signals and/or magnetic resonance signals for magnetic resonance examinations in combination with a positron emission tomography examination (PET examination) conducted on a patient. A difficulty that exists here, however, is that the local magnetic resonance radiofrequency antenna units, in particular flexible magnetic resonance radiofrequency antenna units, can be arranged at different positions on the patient and consequently the local magnetic resonance radiofrequency antenna units prove unsatisfactory for the generation of an attenuation value map for the PET examination.

When magnetic resonance examinations are combined with PET examinations, however, a maximally precise knowledge of a position and/or an arrangement and/or a radius of curvature of the local magnetic resonance radiofrequency antenna unit is necessary in order to accurately determine a signal attenuation experienced by photons of a PET examination when passing through matter, in particular the local magnetic resonance radiofrequency antenna unit. If no allowance is made for attenuation corrections, this can lead to missing PET events in the PET data and/or to image artifacts in the reconstructed image data.

Flexible magnetic resonance radiofrequency antenna units according to the prior art are constructed in such a way that photons experience an absolute minimum of attenuation when passing through. Owing to the low attenuation, no attention has hitherto been given to allowing for the local magnetic resonance radiofrequency antenna unit in an attenuation correction.

### SUMMARY

At least one embodiment of the present invention is in particular to provide a magnetic resonance radiofrequency antenna unit which can be taken into account in an attenuation correction of a PET measurement. Advantageous embodiments are described in the dependent claims.

At least one embodiment of the invention is based on a magnetic resonance radiofrequency antenna unit having at least one antenna element and an antenna housing enclosing the antenna element.

It is proposed that the magnetic resonance radiofrequency antenna unit have at least one marking element, the at least one marking element being embodied so as to transmit magnetic resonance signals during a magnetic resonance measurement. The embodiment according to the invention enables precise detection of a position of the magnetic resonance radiofrequency antenna unit during the magnetic resonance measurement, thus enabling accurate attenuation values to be provided for a positron emission tomography examination (PET examination) on the basis of the precise position of the magnetic resonance radiofrequency antenna unit. Furthermore, interpretation errors in an evaluation of positron emission tomography data can be reduced and/or prevented owing to the accurate attenuation values and consequently an improvement in the image quality of the evaluated PET data can be achieved.

At least one embodiment of the invention is furthermore based on a combined imaging system, comprising: a positron emission tomography device; and a magnetic resonance device including a magnet unit comprising a main magnet, a gradient coil unit and a radiofrequency antenna unit, and a local magnetic resonance radiofrequency antenna unit including at least one marking element, wherein the at least one marking element is embodied to transmit magnetic resonance signals during a magnetic resonance measurement.

At least one embodiment of the invention is furthermore based on a method for calculating attenuation values of a local magnetic resonance radiofrequency antenna unit for a magnetic resonance examination combined with a positron emission tomography examination, the method comprising:

transmitting a magnetic resonance sequence for the purpose of the resonant excitation of atomic nuclei of at least one marking element of a local magnetic resonance radiofrequency antenna unit;

acquiring magnetic resonance signals transmitted by the atomic nuclei of the at least one marking element by way of at least one of the local magnetic resonance radiofrequency antenna unit and a magnet unit of a magnetic resonance device;

determining at least one of a position and location of the local magnetic resonance radiofrequency antenna unit on the basis of the acquired magnetic resonance signals transmitted by the at least one marking element; and

calculating attenuation values of the local magnetic resonance radiofrequency antenna unit on the basis of the determined at least one of position and location of the local magnetic resonance radiofrequency antenna unit for generation of an attenuation value map for the positron emission tomography examination.

At least one embodiment of the invention is furthermore based on a computer program which can be loaded directly into a memory of a programmable evaluation unit of the combined imaging system, the computer program comprising program segments for performing a method for calculating attenuation values of a local magnetic resonance radiofrequency antenna unit for a magnetic resonance examination combined with a PET examination when the computer program is executed in the evaluation unit of the combined imaging system. A realization in software of this kind has the advantage that known evaluation units of combined imaging systems comprising a magnetic resonance device with integrated positron emission tomography device can be modified through implementation of the computer program in a suitable manner in order to calculate attenuation values of a local magnetic resonance radiofrequency antenna unit according to at least one embodiment of the inventive method for an attenuation value map of a magnetic resonance examination combined with a positron emission tomography examination.

### BRIEF DESCRIPTION OF THE DRAWINGS

Further advantages, features and details of the invention will become apparent from the example embodiment described hereinbelow as well as with reference to the drawings, in which:

FIG. 1 shows a combined imaging system according to an embodiment of the invention having a local magnetic resonance radiofrequency antenna unit in a schematic representation,

FIG. 2 shows the local magnetic resonance radiofrequency antenna unit in a schematic representation,

FIG. 3 shows the local magnetic resonance radiofrequency antenna unit in a sectional representation,

FIG. 4 shows an alternative embodiment of the local magnetic resonance radiofrequency antenna unit in a sectional representation, and

FIG. 5 is a flowchart of a method for calculating attenuation values of a local radiofrequency antenna unit.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Methods discussed below, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks will be stored in a machine or computer readable medium such as a storage medium or non-transitory computer readable medium. A processor(s) will perform the necessary tasks.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments may be described with reference to acts and symbolic representations of operations (e.g., in the form of flowcharts) that may be implemented as program modules or functional processes include routines, programs, objects, components, data structures, etc., that perform particular tasks or implement particular abstract data types and may be implemented using existing hardware at existing network elements. Such existing hardware may include one or more Central Processing Units (CPUs), digital signal processors (DSPs), application-specific-integrated-circuits, field programmable gate arrays (FPGAs) computers or the like.

Note also that the software implemented aspects of the example embodiments may be typically encoded on some form of program storage medium or implemented over some type of transmission medium. The program storage medium (e.g., non-transitory storage medium) may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The example embodiments not limited by these aspects of any given implementation.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

It is proposed that the magnetic resonance radiofrequency antenna unit have at least one marking element, the at least one marking element being embodied so as to transmit magnetic resonance signals during a magnetic resonance measurement. The embodiment according to the invention enables precise detection of a position of the magnetic resonance radiofrequency antenna unit during the magnetic resonance measurement, thus enabling accurate attenuation values to be provided for a positron emission tomography examination (PET examination) on the basis of the precise position of the magnetic resonance radiofrequency antenna unit. Furthermore, interpretation errors in an evaluation of positron emission tomography data can be reduced and/or prevented owing to the accurate attenuation values and consequently an improvement in the image quality of the evaluated PET data can be achieved.

What is to be understood by a magnetic resonance radiofrequency antenna unit in this context is in particular a local magnetic resonance radiofrequency antenna unit which is arranged directly or at a minimum distance around a region of the patient's body that is to be examined and which is configured in particular for receiving magnetic resonance signals and/or transmitting excitation signals. A position of the local magnetic resonance radiofrequency antenna unit within a patient examination area of a medical imaging system is in this case dependent on the region of the body that is to be examined and/or on the patient's anatomy.

Preferably the local magnetic resonance radiofrequency antenna unit is arranged together with a patient inside a patient receiving zone of a combined imaging system comprising a magnetic resonance device and a positron emission tomography device (PET device) integrated into the magnetic resonance device for the purpose of a pending medical imaging examination, the local magnetic resonance radiofrequency antenna unit being embodied separately from a radiofrequency antenna unit that is permanently integrated within a magnet unit of the magnetic resonance device. The marking element is preferably formed from a material whose atomic nuclei are excited by way of a magnetic resonance sequence and subsequently transmit magnetic resonance signals for the purpose of localizing the local magnetic resonance radiofrequency antenna unit.

It is furthermore proposed that the at least one marking element comprise a material whose atomic nuclei have an excitation frequency that is different from the excitation frequency of hydrogen atoms. This enables undesirable signal convolutions due to the marking elements to be advantageously reduced and/or prevented in the data of a medical magnetic resonance examination. Moreover, the acquisition of magnetic resonance signals for the purpose of determining the position of the local magnetic resonance radiofrequency antenna unit can take place free of interference and/or independently of the acquisition of medical magnetic resonance data of a medical magnetic resonance examination. The excitation frequency is preferably formed from the Larmor frequency, the Larmor frequency of hydrogen atoms, in particular of a proton, having a value of 42.58 MHz.

The at least one marking element particularly advantageously comprises a material whose atomic nuclei have an excitation frequency which has a maximum separation of 14 MHz from the excitation frequency of hydrogen atoms. In this way a particularly advantageous sensitivity can be achieved for the acquisition of magnetic resonance signals of the marking element, without the necessity in this case to adjust a bandwidth in the setting of gradient strengths and/or gradient pulses. In this context the closer the excitation frequency of atoms of the marking element is to the excitation frequency and/or Larmor frequency of hydrogen atoms, the higher is a sensitivity of hardware configured for acquiring magnetic resonance signals, in particular a sensitivity of radiofrequency antenna units and/or of a gradient system, for the acquisition of the magnetic resonance signals transmitted by the marking element. This can also lead to a particularly time-saving acquisition of the magnetic resonance signals transmitted by the marking element. The atomic nuclei of the marking element advantageously have an excitation frequency having a maximum separation of 7 MHz and particularly preferably a maximum separation of 3 MHz from the excitation frequency of hydrogen atoms.

In an advantageous development of at least one embodiment of the invention it is proposed that at least one marking unit comprise a material whose atomic nuclei have a spin having a value=½. In this case acquired magnetic resonance signals can be uniquely assigned to the marking element, since only one energy level of the atoms is available for an excitation of the atoms.

The at least one marking element particularly advantageously comprises a fluorine material. Preferably the at least one marking element comprises a material containing ¹⁹fluorine (¹⁹F). A ¹⁹F material has the advantage that it has both a spin having a value of ½ and also an excitation frequency of the ¹⁹F material very close to the excitation frequency of hydrogen atoms. The excitation frequency of a ¹⁹F material is in the region of 40.05 MHz. Furthermore, other materials, such as ³¹P having an excitation frequency of 17.24 MHz or ⁷Li having an excitation frequency of 16.55 MHz or ²³Na having an excitation frequency of 11.42 MHz, for example, and/or further materials appearing as suitable to the person skilled in the art, can be used in the embodiment of the marking element.

In an advantageous development of at least one embodiment of the invention it is proposed that the magnetic resonance radiofrequency antenna unit have a plurality of marking elements that are arranged spaced apart from one another. Preferably the magnetic resonance radiofrequency antenna unit has at least three marking elements so that a position and/or a volume of the magnetic resonance radiofrequency antenna unit can be unequivocally identified by way of the magnetic resonance data transmitted by the marking elements. Preferably the different marking elements are arranged on the antenna housing of the magnetic resonance radiofrequency antenna unit. Alternatively or in addition the marking element can also be formed by a marking layer, wherein the layer can preferably be arranged on the antenna housing. Other embodiment variants of the marking elements are conceivable at any time.

At least one embodiment of the invention is furthermore based on a combined imaging system comprising a magnetic resonance device and a positron emission tomography device, wherein the magnetic resonance device has a magnet unit comprising a basic field magnet, a gradient coil unit and a radiofrequency antenna unit, and a local magnetic resonance radiofrequency antenna unit having at least one marking element, wherein the at least one marking element is embodied so as to transmit magnetic resonance signals during a magnetic resonance measurement. This enables precise detection of a position of the magnetic resonance radiofrequency antenna unit during the magnetic resonance measurement such that accurate attenuation values can be provided for a positron emission tomography examination (PET examination) on the basis of the precise position of the magnetic resonance radiofrequency antenna unit. Furthermore, interpretation errors in an evaluation of positron emission tomography data can be reduced and/or prevented on account of the accurate attenuation values and consequently an improvement in the image quality of the evaluated PET data can be achieved.

It is furthermore proposed that the local magnetic resonance radiofrequency antenna unit and/or the radiofrequency antenna unit of the magnet unit have a plurality of transmit channels for transmitting radiofrequency pulses, wherein one transmit channel of the plurality of transmit channels is configured for transmitting a radiofrequency pulse for exciting atomic nuclei of the marking element.

Alternatively or in addition, the local magnetic resonance radiofrequency antenna unit and/or the radiofrequency antenna unit of the magnet unit have/has a plurality of receive channels, wherein one receive channel of the plurality of receive channels is configured for acquiring magnetic resonance signals of the marking element. In this way a parallel, i.e. simultaneous, acquisition of medical magnetic resonance signals and magnetic resonance signals transmitted by the marking element and provided for localizing the local magnetic resonance radiofrequency antenna unit can be achieved in a particularly time-saving manner. For this purpose the local magnetic resonance radiofrequency antenna unit and/or the radiofrequency antenna unit of the magnet unit preferably comprise/comprises a dual-resonant antenna unit having, for example, two separate preamplifiers for the two different signals. Furthermore, the other transmit channels and/or receive channels of the local magnetic resonance radiofrequency antenna unit and/or of the radiofrequency antenna unit of the magnet unit are configured for transmitting radiofrequency pulses for the medical magnetic resonance examination and/or for receiving medical magnetic resonance signals.

A fast and direct determination of attenuation values for the PET examination can be achieved if the combined imaging system has an evaluation unit which is configured for calculating attenuation values of the local magnetic resonance radiofrequency antenna unit on the basis of the magnetic resonance signals transmitted by the at least one marking element.

At least one embodiment of the invention is furthermore based on a method for calculating attenuation values of a local magnetic resonance radiofrequency antenna unit for a magnetic resonance examination combined with a positron emission tomography examination, the method comprising:

transmitting a magnetic resonance sequence for the purpose of the resonant excitation of atomic nuclei of at least one marking element of a local magnetic resonance radiofrequency antenna unit;

acquiring magnetic resonance signals transmitted by the atomic nuclei of the at least one marking element by way of at least one of the local magnetic resonance radiofrequency antenna unit and a magnet unit of a magnetic resonance device;

determining at least one of a position and location of the local magnetic resonance radiofrequency antenna unit on the basis of the acquired magnetic resonance signals transmitted by the at least one marking element; and

calculating attenuation values of the local magnetic resonance radiofrequency antenna unit on the basis of the determined at least one of position and location of the local magnetic resonance radiofrequency antenna unit for generation of an attenuation value map for the positron emission tomography examination.

The method according to at least one embodiment of the invention enables a particularly time-saving and accurate determination of attenuation values of the magnetic resonance radiofrequency antenna unit or, as the case may be, an accurate determination of an attenuation value map for the positron emission tomography examination (PET examination) and consequently an improvement in the image quality of the evaluated PET data of the combined magnetic resonance examination. Furthermore, interpretation errors in an evaluation of positron emission tomography data of the combined magnetic resonance examination can be reduced and/or prevented on account of the accurate attenuation values.

It is furthermore proposed that the transmission of radiofrequency pulses for the purpose of the resonant excitation of atomic nuclei of the at least one marking element of the local magnetic resonance radiofrequency antenna unit take place simultaneously with a transmission of radiofrequency pulses of the medical magnetic resonance examination and/or the acquisition of the magnetic resonance signals transmitted by the atoms of the at least one marking element take place simultaneously with an acquisition of magnetic resonance signals of the medical magnetic resonance examination. By this, a particularly short overall examination time can be achieved for the medical imaging examination and consequently the length of time that the patient spends in a patient receiving zone of the magnetic resonance device with integrated PET device can be reduced.

Alternatively hereto, radiofrequency pulses for the purpose of the resonant excitation of atomic nuclei of the at least one marking element of the local magnetic resonance radiofrequency antenna unit can be transmitted during measurement pauses in the medical magnetic resonance examination and/or the magnetic resonance signals transmitted by the atomic nuclei of the at least one marking element can be acquired during measurement pauses in the medical magnetic resonance examination. By this, an undesirable degradation and/or interference, such as for instance a coupling between different receive channels and/or transmit channels of the local magnetic resonance radiofrequency antenna unit and/or of the radiofrequency antenna unit of the magnet unit, during the transmission of radiofrequency signals and/or during the acquisition of the magnetic resonance signals can advantageously be prevented. The transmission of radiofrequency pulses for the purpose of the resonant excitation of atomic nuclei of the at least one marking element of the local magnetic resonance radiofrequency antenna unit and the acquisition of the magnetic resonance signals transmitted by the atomic nuclei of the at least one marking element can also take place prior to the medical magnetic resonance examination, for example in the form of an overview measurement and/or a scout measurement.

In addition it is proposed that a bandwidth of a gradient coil unit be adjusted to an excitation frequency of atomic nuclei of the marking element. In this way a sensitivity for the acquisition of the magnetic resonance signals of the atomic nuclei of the marking element can advantageously be increased and consequently a measurement time for the acquisition of the magnetic resonance signals can be significantly shortened. Furthermore, the length of time that the patient spends in a patient receiving zone of the magnetic resonance device with integrated PET device can be reduced as a result.

At least one embodiment of the invention is furthermore based on a computer program which can be loaded directly into a memory of a programmable evaluation unit of the combined imaging system, the computer program comprising program segments for performing a method for calculating attenuation values of a local magnetic resonance radiofrequency antenna unit for a magnetic resonance examination combined with a PET examination when the computer program is executed in the evaluation unit of the combined imaging system. A realization in software of this kind has the advantage that known evaluation units of combined imaging systems comprising a magnetic resonance device with integrated positron emission tomography device can be modified through implementation of the computer program in a suitable manner in order to calculate attenuation values of a local magnetic resonance radiofrequency antenna unit according to at least one embodiment of the inventive method for an attenuation value map of a magnetic resonance examination combined with a positron emission tomography examination.

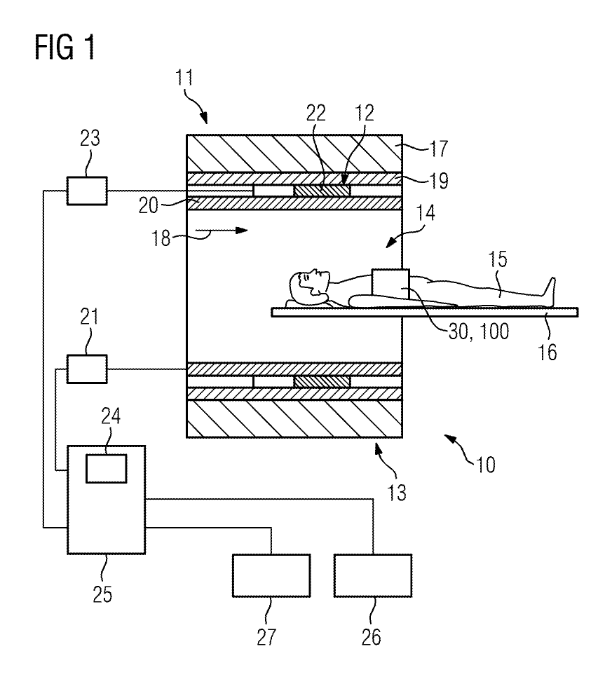

FIG. 1 shows a combined medical imaging system 10. The combined medical imaging system 10 comprises a magnetic resonance device 11 and a positron emission tomography device 12 (PET device 12).

The magnetic resonance device 11 comprises a magnet unit 13 and a patient receiving zone 14 enclosed by the magnet unit 13 and intended for receiving a patient 15, the patient receiving zone 14 being cylindrically surrounded by the magnet unit 13 in a circumferential direction. The patient 15 can be introduced into the patient receiving zone 14 by way of a patient positioning device 16 of the magnetic resonance device 11. The patient positioning device 16 is movably arranged inside the patient receiving zone 14 for this purpose.

The magnet unit 13 comprises a main magnet 17 which is designed to generate a strong and in particular constant main magnetic field 18 during the operation of the magnetic resonance device 11. The magnet unit 13 additionally has a gradient coil unit 19 for generating magnetic field gradients which is used for spatial encoding during imaging. The magnet unit 13 furthermore comprises a first radiofrequency antenna unit 20 which is formed by a radiofrequency antenna transmit unit and which is configured for the purpose of exciting a polarization which becomes established in the main magnetic field 18 generated by the main magnet 17. The first radiofrequency antenna unit 20 is permanently integrated within the magnet unit 13.

In order to control the main magnet 17 of the gradient coil unit 19 and in order to control the radiofrequency antenna unit 20, the medical imaging system 10, in particular the magnetic resonance device 11, has a control unit 21 formed by a computing unit. The control unit 21 centrally controls the magnetic resonance device 11, such as in order to execute a predetermined imaging gradient echo sequence for example. For this purpose the control unit 21 comprises a gradient control unit (not shown in any further detail) and a radiofrequency antenna control unit (not shown in any further detail). The control unit 21 additionally comprises an evaluation unit (not shown in any further detail) for evaluating magnetic resonance image data.

The illustrated magnetic resonance device 11 can of course comprise further components that are typically included in magnetic resonance devices. The general mode of operation of a magnetic resonance device 11 is furthermore well-known to the person skilled in the art, so a detailed description of the general components will be dispensed with.

The PET device 12 comprises a plurality of positron emission tomography detector modules 22 (PET detector modules 22) which are arranged in a ring shape and surround the patient receiving zone 14 in the circumferential direction. In this case the PET detector modules 22 are arranged between the radiofrequency antenna unit 20 and the gradient coil unit 19 of the magnetic resonance device 11 and thus are integrated into the magnetic resonance device 11 in a particularly space-saving manner.

The PET detector modules 22 each have a plurality of positron emission tomography detector elements (PET detector elements) (not shown in any further detail) which are arranged to form a PET detector array comprising a scintillation detector array having scintillation crystals, for example LSO crystals. In addition the PET detector modules 22 each comprise a photodiode array, for example an avalanche photodiode array or APD photodiode array, which are arranged downstream of the scintillation detector array inside the PET detector modules 22. The PET detector array also includes detector electronics (not shown in any further detail) comprising an electric amplifier circuit and further electronic components which are not shown in further detail.

In order to control the PET detector modules 22, the PET device 12 has a control unit 23. The PET device 12 shown can of course comprise further components that are typically included in PET devices. The general mode of operation of a PET device 12 is furthermore well-known to the person skilled in the art, so a detailed description of the general components will be dispensed with.

Photon pairs resulting from the annihilation of a positron with an electron are detected by way of the PET detector modules 22. Trajectories of the two photons enclose an angle of 1800. Furthermore, the two photons each have an energy of 511 keV. In this case the positron is emitted by a radiopharmaceutical, the radiopharmaceutical being administered to the patient 15 by way of an injection. When passing through matter, the photons produced in the annihilation can be absorbed, the absorption probability being dependent on the path length through the matter and the corresponding absorption coefficient of the matter. Accordingly, in an evaluation of positron emission tomography signals (PET signals) it is necessary to correct the signals in respect of their attenuation by components that are located in the beam path. Toward that end an attenuation value map for the evaluation of the PET signals is produced on the basis of the components.

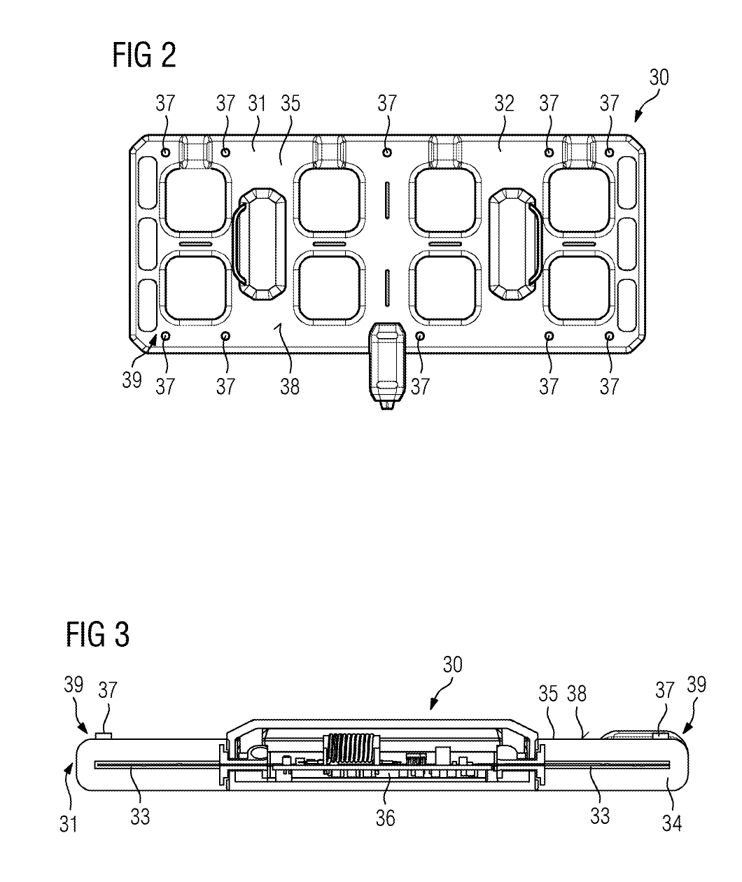

One of the components can be for example a local magnetic resonance radiofrequency antenna unit 30 which, for the purpose of acquiring the magnetic resonance signals, is applied around a body region of the patient 15 that is to be examined (FIG. 1). The local magnetic resonance radiofrequency antenna unit 30 is incorporated in the magnetic resonance device 11. The local magnetic resonance radiofrequency antenna unit 30 is shown in more detail in FIGS. 2 and 3 and comprises a plurality of antenna sections 31, 32, each of which has an antenna element 33, a stabilization layer 34, and an antenna housing 35 enclosing the antenna element 33 and the stabilization layer 34. In addition the local magnetic resonance radiofrequency antenna unit 30 comprises antenna electronics 36 likewise enclosed by the antenna housing 35. Furthermore, each of the antenna sections 31, 32 can have a carrier layer on which the antenna element 33 is arranged.

In the present exemplary embodiment the local magnetic resonance radiofrequency antenna unit 30 is formed by a whole-body radiofrequency antenna unit which is preferably positioned around a chest region of the patient 15 for the purpose of a magnetic resonance examination (FIG. 1). Alternatively hereto, further embodiments of the local magnetic resonance radiofrequency antenna unit 30 are conceivable at any time, such as an embodiment of the local magnetic resonance radiofrequency antenna unit 30 as, for example, a head coil unit, knee coil unit, etc.

The local magnetic resonance radiofrequency antenna unit 30 of FIGS. 2 and 3 additionally has a plurality of marking elements 37. The individual marking elements 37 are embodied in such a way that magnetic resonance signals are transmitted by the marking elements 37 during a magnetic resonance measurement in order to enable the local magnetic resonance radiofrequency antenna unit 30 to be localized on the basis of the acquired magnetic resonance signals.

In medical magnetic resonance examinations, in particular an excitation frequency is transmitted in order to excite hydrogen atoms in the human body. The excitation frequency is preferably formed by the Larmor frequency, the Larmor frequency of hydrogen atoms, in particular of protons, having a value of 42.58 MHz. However, the marking elements 37 comprise a material whose atomic nuclei preferably have an excitation frequency which is different from an excitation frequency of hydrogen atoms, such that a unique assignment of the different magnetic resonance signals to the marking elements 37 or, as the case may be, to the hydrogen atoms of the human body can be achieved. However, the smaller a separation of the excitation frequency of the atomic nuclei of the material of the marking elements 37 of the local magnetic resonance radiofrequency antenna unit 30 is from the excitation frequency of the atomic nuclei of the hydrogen atoms, the higher in this case is a sensitivity of the hardware components, in particular the gradient coil unit 19, the radiofrequency antenna unit 20 and the local magnetic resonance radiofrequency antenna unit 30, of the magnetic resonance device 11 for receiving and/or acquiring the magnetic resonance signals transmitted by the atomic nuclei of the marking elements 27. Preferably the excitation frequency of the atomic nuclei of the marking elements 37 has a maximum separation of 14 MHz, preferably a maximum separation of 7 MHz, and particularly preferably a maximum separation of 3 MHz, from the excitation frequency of hydrogen atoms.

The marking elements 37 additionally comprise a material whose atomic nuclei have a spin with value=½, such that only a single excitation frequency is available for the atomic nuclei and consequently a unique assignment of the measurement results is achieved in an evaluation of the magnetic resonance signals emitted by the marking elements 37.

In the present exemplary embodiment (FIGS. 2 and 3) the material of the individual marking elements 37 comprises a ¹⁹fluorine material. The excitation frequency of ¹⁹fluorine materials is in the region of 40.05 MHz and therefore is very close to the excitation frequency of hydrogen atoms. Furthermore, other materials, such as ³¹P having an excitation frequency of 17.24 MHz, for example, or ⁷Li having an excitation frequency of 16.55 MHz and/or further materials appearing suitable to the person skilled in the art can be used in the embodiment of the marking elements 37, although the materials of the marking elements 37 necessitate an adjustment of a bandwidth of the gradient coil unit 19 so that an advantageous sensitivity is given in respect of the magnetic resonance signals transmitted by the marking elements 37.

The individual marking elements 37 of the local magnetic resonance radiofrequency antenna unit 30 are furthermore arranged spaced apart from one another, as can be seen from FIGS. 2 and 3. In the present exemplary embodiment the individual marking elements 37 are arranged spaced apart from one another on an external surface 38 of the antenna housing 35 of the local magnetic resonance radiofrequency antenna unit 30, such that an extent and/or a position and/or a location of the local magnetic resonance radiofrequency antenna unit 30 can be unequivocally determined from the acquired magnetic resonance signals transmitted by the marking elements 37.

For the unequivocal and/or exact detection of the extent and/or position and/or location of the local magnetic resonance radiofrequency antenna unit 30, the local magnetic resonance radiofrequency antenna unit 30 preferably has three or more marking elements 37 arranged in a distributed manner. In the present exemplary embodiment the individual marking elements 37 are embodied in a circular shape, preferably being arranged at boundary regions 39 of the local magnetic resonance radiofrequency antenna unit 30. Alternatively hereto, the individual marking elements 37 can also be embodied as punctiform and/or have other geometric embodiments appearing suitable to the person skilled in the art, such as a rectangular or lamellar embodiment, for example.

The individual marking elements 37 can furthermore also be arranged as removable on the external surface 38 of the antenna housing 35, such that the local magnetic resonance radiofrequency antenna unit 30 can be used without marking elements 37 if the unit 30 is used exclusively for magnetic resonance measurements. In another embodiment of the local magnetic resonance radiofrequency antenna unit 30 it is furthermore also conceivable for the individual marking elements 37 to be arranged within an area of the local magnetic resonance radiofrequency antenna unit 30 that is enclosed by the antenna housing 35.

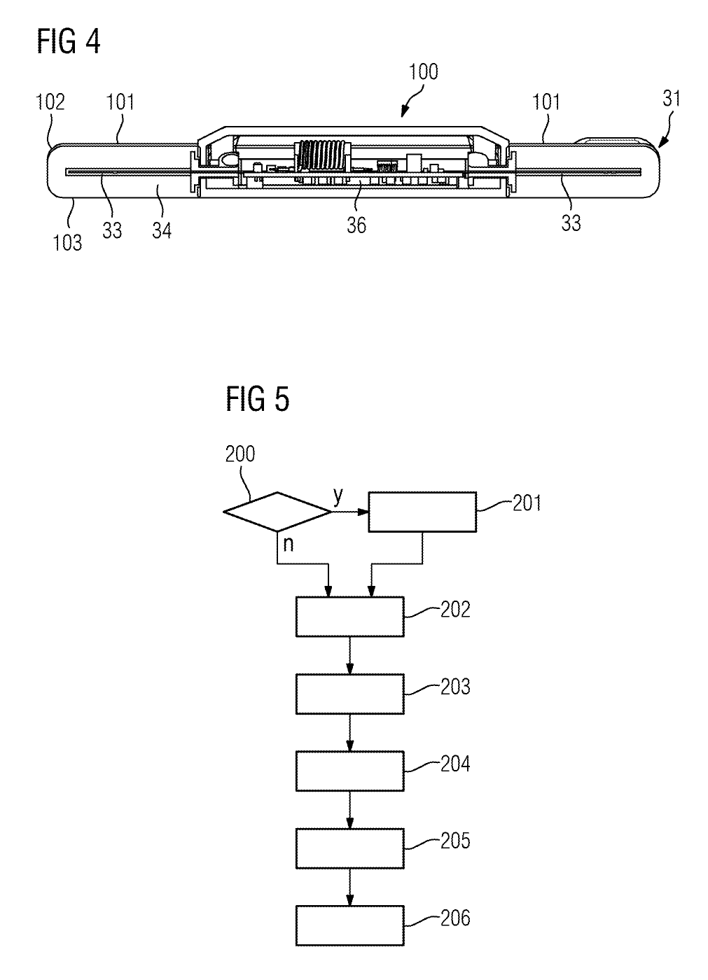

An alternative embodiment of the local magnetic resonance radiofrequency antenna unit to FIGS. 2 and 3 is shown in FIG. 4. Components, features and functions remaining substantially the same are labeled consistently with the same reference numerals. The following description restricts itself essentially to the differences from the exemplary embodiment in FIGS. 2 and 3, with reference being made to the description of the exemplary embodiment in FIGS. 2 and 3 in respect of components, features and functions that remain the same.

The local magnetic resonance radiofrequency antenna unit 100 differs from the local magnetic resonance radiofrequency antenna unit 20 of FIGS. 2 and 3 in terms of an embodiment, number and arrangement of marking elements 37, 101.

The local magnetic resonance radiofrequency antenna unit 100 in FIG. 4 has a single marking element 101 which is formed by a marking element layer. In the present exemplary embodiment the marking element layer is arranged on an external surface 102 of an antenna housing 103 of the local magnetic resonance radiofrequency antenna unit 100. In principle the marking element layer could also be arranged within an area of the local magnetic resonance radiofrequency antenna unit 100 enclosed by the antenna housing 103.

The further embodiments of the marking element 101, in particular in terms of material properties of the marking element 101, correspond to the statements made with reference to FIGS. 2 and 3. Furthermore, another embodiment of the local magnetic resonance radiofrequency antenna unit 100 also corresponds to the statements made with reference to FIGS. 2 and 3.

For the purpose of a magnetic resonance measurement the local magnetic resonance radiofrequency antenna unit 30, 100 and/or the radiofrequency antenna unit 20 of the magnet unit 13 have/has a plurality of transmit channels for transmitting radiofrequency pulses, one transmit channel of the plurality of transmit channels being configured for transmitting a radiofrequency pulse for the purpose of exciting atomic nuclei of the marking elements 37, 101. Furthermore, the local magnetic resonance radiofrequency antenna unit 30, 100 and/or the radiofrequency antenna unit 20 of the magnet unit 13 have/has a plurality of receive channels, one receive channel of the plurality of receive channels being configured for acquiring magnetic resonance signals of the marking elements 37, 101. Moreover, the further transmit channels and/or receive channels of the local magnetic resonance radiofrequency antenna unit 30, 100 and/or of the radiofrequency antenna unit 20 of the magnet unit 11 are configured for transmitting radiofrequency pulses for the medical magnetic resonance examination and/or for receiving medical magnetic resonance signals. In this arrangement the local magnetic resonance radiofrequency antenna unit 30, 100 and/or the radiofrequency antenna unit 20 of the magnet unit 13 are/is embodied as dual-resonant antenna units having for example two separate preamplifiers for the two different signals. This embodiment of the local magnetic resonance radiofrequency antenna unit 30, 100 and/or of the radiofrequency antenna unit 20 of the magnet unit 13 enables a simultaneous transmission of radiofrequency pulses for the purpose of the resonant excitation of atomic nuclei of the marking elements 37, 101 of the local magnetic resonance radiofrequency antenna unit 30, 101 and of radiofrequency pulses of the medical magnetic resonance examination. In addition this embodiment of the local magnetic resonance radiofrequency antenna unit 30, 101 and/or of the radiofrequency antenna unit 20 of the magnet unit 13 enables a simultaneous acquisition of the magnetic resonance signals transmitted by the atoms of the marking elements 37, 101 and of signals of the medical magnetic resonance examination.

For an evaluation of the magnetic resonance signals transmitted by the marking elements 37, 101, the combined imaging system 10 has an evaluation unit 24 which is integrated inside a system control unit 25 of the combined imaging system 10 (FIG. 1). The system control unit 25 for example synchronizes the magnetic resonance examination and the PET examination for the purpose of a combined examination.

The evaluation unit 24 of the combined imaging system 10 is configured for calculating attenuation values of the local magnetic resonance radiofrequency antenna unit 30, 100 on the basis of the magnetic resonance signals transmitted by the marking elements 37, 101 and for making an attenuation value map available for an evaluation of the PET signals. Data is transmitted between the individual components and subcomponents of the combined imaging system 10 by way of a data transmission unit which is not shown in any further detail (FIG. 1).

The combined imaging system additionally comprises a display unit 26, for example a monitor, for displaying control information such as, for example, imaging parameters for an operator of the combined imaging system 10. The combined imaging system 10 also has an input unit 27 by which information and/or parameters can be input by an operator during a measurement procedure.

The combined imaging system 10 shown can of course comprise other components that are typically included in combined imaging systems. The general mode of operation of a combined imaging system 10 is furthermore well-known to the person skilled in the art, so a detailed description of the general components will be dispensed with.

FIG. 5 shows a flowchart of an embodiment of an inventive method for calculating attenuation values of a local magnetic resonance radiofrequency antenna unit 30, 100 for a magnetic resonance examination combined with a positron emission tomography examination (PET examination).

Firstly, it is queried by way of the evaluation unit 24 in a first method step 200 whether an adjustment of a bandwidth of the gradient coil unit 19 is necessary. Depending on the embodiment of the marking elements 37, 101 of the local magnetic resonance radiofrequency antenna unit 30, 100, a bandwidth of the gradient coil unit 19 is adjusted to an excitation frequency of the atomic nuclei of the marking elements 37, 101 in a further method step 201 by way of the evaluation unit 24. If the marking elements 37, 101 include a ¹⁹fluorine material, this method step can be omitted (n) since the excitation frequency of ¹⁹fluorine materials is in the region of 40.05 MHz and consequently is very close to the excitation frequency of hydrogen atoms, which have an excitation frequency of 42.58 MHz. If, on the other hand, the marking elements 37, 101 include materials containing for example ³¹P or ⁷Li, an adjustment of the bandwidth of the gradient coil unit 19 is necessary (y) in order to increase a sensitivity of the hardware components during a magnetic resonance measurement for the purpose of acquiring the magnetic resonance signals transmitted by the marking elements 37, 101. In this case material properties of the marking elements 37, 101 can be detected automatically by way of the magnet unit 13 or communicated to the system by way of a manual input by a member of the medical operating staff.

Following the query and, where applicable, after method step 201 of adjusting the bandwidth, a magnetic resonance sequence is transmitted in a further method step 202 for the purpose of the resonant excitation of atomic nuclei of the marking elements 37, 101 of the local magnetic resonance radiofrequency antenna unit 30, 100. The magnetic resonance sequence is transmitted by way of the radiofrequency antenna unit 20 of the magnet unit 13 and/or by way of the local magnetic resonance radiofrequency antenna unit 30, 100. The radiofrequency pulses intended for the resonant excitation of the atomic nuclei of the marking elements 37, 101 of the local magnetic resonance radiofrequency antenna unit 30, 100 are preferably transmitted simultaneously with a transmission of radiofrequency pulses of the medical magnetic resonance examination. Furthermore, it is also conceivable for the transmission of the radiofrequency pulses for the purpose of the resonant excitation of the atomic nuclei of the marking elements 37, 101 of the local magnetic resonance radiofrequency antenna unit 30, 100 to take place during measurement pauses in the medical magnetic resonance examination or prior to the medical magnetic resonance examination, for example in the form of an overview measurement and/or a scout measurement.

Following on therefrom, in a further method step 203, magnetic resonance signals transmitted by the atomic nuclei of the marking elements 37, 101 are acquired by way of the local magnetic resonance radiofrequency antenna unit 30, 100 and/or the radiofrequency antenna unit 20 of the magnet unit 13. The magnetic resonance signals transmitted by the atoms of the marking elements 37, 101 are likewise preferably acquired simultaneously with an acquisition of signals of the medical magnetic resonance examination. Furthermore, it is also conceivable for the acquisition of the magnetic resonance signals transmitted by the atomic nuclei of the marking elements 37, 101 to take place during measurement pauses in the medical magnetic resonance examination or prior to the medical magnetic resonance examination.

In a further method step 204, a position and/or location of the local magnetic resonance radiofrequency antenna unit 30, 100 inside the patient receiving zone 14 are/is determined by way of the evaluation unit 24 of the combined imaging system 10 on the basis of the acquired magnetic resonance signals. If the magnetic resonance signals transmitted by the atomic nuclei of the marking elements 37, 101 are acquired simultaneously with the magnetic resonance signals of the medical magnetic resonance examination, the different magnetic resonance signals are furthermore first separated by the evaluation unit 24 in this method step 204 and only then are/is the position and/or location of the local magnetic resonance radiofrequency antenna unit 30, 100 determined on the basis of the magnetic resonance signals transmitted by the marking elements 37, 101.

Following on therefrom, in a further method step 205, attenuation values for the local magnetic resonance radiofrequency antenna unit 30, 100 are calculated by the evaluation unit 24 on the basis of the position and/or location of the local magnetic resonance radiofrequency antenna unit 30, 100. For this purpose further parameters, such as for instance an absorption probability of a material of the local magnetic resonance radiofrequency antenna unit 30, 100, from a database stored in a memory unit (not shown in any further detail) of the evaluation unit 24 are taken into account by the evaluation unit 24. In a further method step 206 following on therefrom, an attenuation value map for the evaluation of the PET examination or, as the case may be, of the PET data is generated by the evaluation unit 24.

In order to calculate the attenuation values of the local magnetic resonance radiofrequency antenna unit 30, 100 for a magnetic resonance examination combined with a PET examination, the evaluation unit 24 has a processor unit and software and/or computer programs required for the evaluation.

The software and/or computer programs are stored directly in a memory unit (not shown in any further detail) of the programmable evaluation unit 24.

The method for calculating the attenuation values of the local magnetic resonance radiofrequency antenna unit for a magnetic resonance examination combined with a PET examination is performed when the computer programs and/or the software are/is executed in the evaluation unit 24.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Although the invention has been illustrated and described in more detail on the basis of the preferred exemplary embodiment, it is not limited by the disclosed examples and other variations can be derived herefrom by the person skilled in the art without leaving the scope of protection of the invention.

1. A magnetic resonance radiofrequency antenna unit comprising:
at least one antenna element;
an antenna housing enclosing the antenna element; and
at least one marking element, the at least one marking element being embodied to transmit magnetic resonance signals during a magnetic resonance measurement.

2. The magnetic resonance radiofrequency antenna unit of claim 1, wherein the at least one marking element includes a material whose atomic nuclei have an excitation frequency which is different from the excitation frequency of hydrogen atoms.

3. The magnetic resonance radiofrequency antenna unit of claim 1, wherein the at least one marking element comprises a material whose atomic nuclei have an excitation frequency that has a maximum separation of 14 MHz from the excitation frequency of hydrogen atoms.

4. The magnetic resonance radiofrequency antenna unit of claim 1, wherein the at least one marking element comprises a material whose atomic nuclei have a spin with a value=½.

5. The magnetic resonance radiofrequency antenna unit of claim 1, wherein the at least one marking element comprises a fluorine material.

6. The magnetic resonance radiofrequency antenna unit of claim 1, wherein the at least one marking element includes a plurality of marking elements, arranged spaced apart from one another.

7. The magnetic resonance radiofrequency antenna unit of claim 1, wherein the at least one marking element is at least partially formed by a marking element layer.

8. A combined imaging system, comprising:
a positron emission tomography device; and
a magnetic resonance device including
a magnet unit comprising a main magnet, a gradient coil unit and a radiofrequency antenna unit, and
a local magnetic resonance radiofrequency antenna unit including at least one marking element, wherein the at least one marking element is embodied to transmit magnetic resonance signals during a magnetic resonance measurement.

9. The combined imaging system of claim 8, wherein at least one of the local magnetic resonance radiofrequency antenna unit and the radiofrequency antenna unit of the magnet unit include a plurality of transmit channels for transmitting radiofrequency pulses, wherein one transmit channel of the plurality of transmit channels is configured for transmitting a radiofrequency pulse for the purpose of exciting atomic nuclei of the at least one marking element.

10. The combined imaging system of claim 8, wherein at least one of the local magnetic resonance radiofrequency antenna unit and the radiofrequency antenna unit of the magnet unit include a plurality of receive channels for receiving magnetic resonance signals, wherein one receive channel of the plurality of receive channels is configured for acquiring magnetic resonance signals of the at least one marking element.

11. The combined imaging system of claim 8, further comprising:
an evaluation unit, configured to calculate attenuation values of the local magnetic resonance radiofrequency antenna unit on the basis of the magnetic resonance signals transmitted by the at least one marking element.

12. A method for calculating attenuation values of a local magnetic resonance radiofrequency antenna unit for a magnetic resonance examination combined with a positron emission tomography examination, the method comprising:
transmitting a magnetic resonance sequence for the purpose of the resonant excitation of atomic nuclei of at least one marking element of a local magnetic resonance radiofrequency antenna unit;
acquiring magnetic resonance signals transmitted by the atomic nuclei of the at least one marking element by way of at least one of the local magnetic resonance radiofrequency antenna unit and a magnet unit of a magnetic resonance device;
determining at least one of a position and location of the local magnetic resonance radiofrequency antenna unit on the basis of the acquired magnetic resonance signals transmitted by the at least one marking element; and
calculating attenuation values of the local magnetic resonance radiofrequency antenna unit on the basis of the determined at least one of position and location of the local magnetic resonance radiofrequency antenna unit for generation of an attenuation value map for the positron emission tomography examination.

13. The method of claim 12, wherein the transmission of radiofrequency pulses for the purpose of the resonant excitation of atomic nuclei of the at least one marking element of the local magnetic resonance radiofrequency antenna unit takes place simultaneously with at least one of a transmission of radiofrequency pulses of the medical magnetic resonance examination and the acquisition of the magnetic resonance signals transmitted by the atoms of the at least one marking element takes place simultaneously with an acquisition of signals of the medical magnetic resonance examination.

14. The method of claim 12, wherein at least one of the transmission of radiofrequency pulses for the purpose of the resonant excitation of atomic nuclei of the at least one marking element of the local magnetic resonance radiofrequency antenna unit takes place during measurement pauses in the medical magnetic resonance examination, and the acquisition of the magnetic resonance signals transmitted by the atomic nuclei of the at least one marking element takes place during measurement pauses in the medical magnetic resonance examination.

15. The method of claim 12, wherein a bandwidth of a gradient coil unit is adjusted to an excitation frequency of atomic nuclei of the at least one marking element.

16. A computer program, directly loadable into a memory of a programmable evaluation unit of a combined imaging system, the computer program comprising program segments for performing a method for calculating attenuation values of a local magnetic resonance radiofrequency antenna unit for a magnetic resonance examination combined with a PET examination of claim 12 when the computer program is executed in the evaluation unit of the combined imaging system.

17. The magnetic resonance radiofrequency antenna unit of claim 2, wherein the at least one marking element comprises a material whose atomic nuclei have an excitation frequency that has a maximum separation of 14 MHz from the excitation frequency of hydrogen atoms.

18. The magnetic resonance radiofrequency antenna unit of claim 2, wherein the at least one marking element comprises a material whose atomic nuclei have a spin with a value=½.

19. The magnetic resonance radiofrequency antenna unit of claim 3, wherein the at least one marking element comprises a material whose atomic nuclei have a spin with a value=½.

20. The magnetic resonance radiofrequency antenna unit of claim 2, wherein the at least one marking element includes a plurality of marking elements, arranged spaced apart from one another.

21. The magnetic resonance radiofrequency antenna unit of claim 2, wherein the at least one marking element is at least partially formed by a marking element layer.

22. The combined imaging system of claim 9, wherein at least one of the local magnetic resonance radiofrequency antenna unit and the radiofrequency antenna unit of the magnet unit include a plurality of receive channels for receiving magnetic resonance signals, wherein one receive channel of the plurality of receive channels is configured for acquiring magnetic resonance signals of the at least one marking element.

23. The combined imaging system of claim 9, further comprising:
an evaluation unit, configured to calculate attenuation values of the local magnetic resonance radiofrequency antenna unit on the basis of the magnetic resonance signals transmitted by the at least one marking element.

24. A computer readable medium including program code segments for, when executed on a local magnetic resonance radiofrequency antenna unit for a combined imaging system including a magnetic resonance examination combined with a PET examination, causing an evaluation unit of the combined imaging system to implement the method of claim 12.
